(19) United States
(12) Patent Application Publication
(10) Pub. No.: US 2009/0130218 A1
(43) Pub. Date: May 21, 2009

(54) Association of Oleaginous Substance With a Mixture of at Least Two Cyclodextrins

(75) Inventors: Mohamed Skiba, Montmain (FR); Malika Lahiani, Montmain (FR); Frederic Bounoure, Rouen (FR); Pierre Dechelotte, Rouen (FR)

Correspondence Address: HARNESS, DICKEY & PIERCE, P.L.C., P.O. BOX 828, BLOOMFIELD HILLS, MI, 48303, US

(73) Assignee: IN CYCLO, Rouen (FR)

(21) Appl. No.: 12/225,499

(22) PCT Filed: Mar. 22, 2007
(86) PCT No.: PCT/FR2007/050986

§ 371 Date: Jan. 16, 2009

(30) Foreign Application Priority Data
Mar. 23, 2006 (FR) 0602526

(51) Int. Cl. A61K 31/20; A61K 35/12; A61K 8/92; A61P 9/00; A61K 31/215; A61K 8/97

(52) U.S. Cl. 424/522; 424/725; 514/558; 514/546; 514/560

(57) ABSTRACT

The invention concerns a mixture of inclusion complexes comprising or consisting of (1) at least two different cyclodextrins selected among alpha-, beta- and gamma cyclodextrin and/or derivatives thereof, in particular derivatives thereof modified by primary and/or secondary hydroxyl groups, and (2) at least one oleaginous substance, in particular selected among animal, vegetable or synthetic oils. The invention also concerns a composition comprising or consisting of such a mixture, the use of such a mixture for preparing a medicine and a method for preparing such inclusion complexes.

### CROSS-REFERENCE TO RELATED APPLICATIONS

This application is a National Phase Entry of PCT/FR2007/050986 filed Mar. 22, 2007, which claims priority to French Application No. 06/02526, filed Mar. 23, 2006, both of which are incorporated by reference herein.

### BACKGROUND AND SUMMARY

The present invention relates to the field of cosmetic, pharmaceutical, dietary, pharmafood, nutraceutical and veterinary compositions. More particularly, the invention relates to inclusion complexes of oleaginous substance or substances, in particular fatty acids, and a mixture of cyclodextrins, the compositions comprising them and a method for preparing such complexes. The invention also relates to compositions comprising at least two cyclodextrins and at least one oleaginous substance.

Oleaginous substances and more particularly unsaturated fatty acids may play a very important part in the organism. For example, they may have an influence on:

cellular activity and humoral immunity,

hormonal regulation,

cardiovascular protection, and

the quality of pregnancy and lactation.

In addition, they are structural components of many cellular membranes.

Since an organism, in particular a human organism, may have deficiencies as regards unsaturated fatty acids, it can be useful to give some thereto. However, such fatty acids may have a specific taste and/or smell. Thus, several documents disclose techniques to try to lessen these disadvantages.

Patent FR 2 547 829 proposes a composition containing compounds of unsaturated fatty acids and a type of cyclodextrin, whose role is to stabilize fatty acids and to reduce the smell and the bitterness associated to polyunsaturated fatty acids. Document EP 0 470 452 describes a product comprising gamma-cyclodextrin for complexing an oleaginous substance containing a mixture of EPA and DHA, polyunsaturated fatty acids with various structures. U.S. Pat. Nos. 5,189,149 and 6,878,696 provide a method for encapsulating oils of animal or vegetable origin, rich in polyunsaturated fatty acids and derivatives thereof, using a certain type of cyclodextrin. However, the protection of oils composed of a mixture of various polyunsaturated fatty acids may be insufficient. The previous documents discuss inclusions of unsaturated fatty acids with gamma-cyclodextrin.

As to patent FR 2 850 040, it describes a complex of acid with alpha-cyclodextrin only. However, the inclusion complexes described above may have an insufficient stability, or insufficiently mask the taste and/or the smell of certain types of oleaginous substances, such as fatty acids.

Besides, the methods described may have problems relating to:

the polymerisation of unsaturated, and more particularly polyunsaturated fatty acids,

the cis-trans isomerization of double bonds, and

the peroxydation of unsaturated fatty acids.

Besides, the integration of unsaturated fatty acids in compositions may be difficult because of their immiscibility or low miscibility in water. Therefore, there is a need for inclusion complexes and for methods for obtaining them, making it possible to overcome all or a part of the problems mentioned above.

According to a first aspect, the object of the invention is a mixture of inclusion complexes comprising, or consisting in:

at least two different cyclodextrins selected from alpha-, beta- and gamma-cyclodextrin and/or derivatives thereof, more particularly derivatives thereof modified by primary and/or secondary hydroxyl groups, and

at least one oleaginous substance, more particularly selected among oils of animal, vegetable and synthetic origin.

The mixture of complexes according to the invention may include a content in oleaginous substance greater than or equal to 40% by weight, more particularly greater than or equal to 50% by weight, in particular greater than or equal to 60% by weight, or even greater than or equal to 70% by weight based on the total weight of the complexes. The oleaginous substance may more particularly include, or even be composed of, at least one fatty acid, in particular a saturated and/or unsaturated fatty acid, a corresponding ester or triglyceride, more particularly a mono- or polyunsaturated fatty acid.

“Fatty acids” means, in the present invention, carboxylic acids comprising 6 to 50 carbon atoms, more particularly 10 to 30 carbon atoms, and in particular 12 to 22 carbon atoms. The name of this class of compounds recalls their natural origin, the fats, which are long chain carboxylic acids esters, and more particularly greases of animal or vegetable origin which may be glycerol triesters. “Unsaturated fatty acids” means, according to the present invention, monounsaturated or polyunsaturated fatty acids.

In particular, the fatty acid may originate from a vegetable, animal, or synthetic oil or from a mixture thereof, more particularly fish oil, linseed oil and/or camelina oil, and in particular a fatty acid may originate from an oil selected from the group comprising:

linseed oil, which may include a content in alpha-linolenic acid of approximately 56%,

walnut, rapeseed and soya bean oil, which may comprise a content of alpha-linolenic acid between 8% and 14%,

blackcurrant seed oil, which may include approximately 12 to 24% linoleic acid, 15 to 19% gamma-linolenic acid, as well as 30 to 40% alpha-linolenic acid and 3 to 4% stearidonic acid (omega-3),

camelina oil, which may include 12 to 24% linoleic acid, as well as 30 to 40% alpha-linolenic acid, 10 to 24% oleic acid and 500 to 800 mg/Kg of tocopherol and tocorienol,

corn, sunflower and grape seed oils, which may be very rich, particularly in linoleic acid, and

fish oils, which may contain high proportions of eicosapentaenoic acid (EPA) and docosahexaenoic acid (DHA).

The oleaginous substance may include a content in unsaturated fatty acid greater than or equal to 30% by weight, particularly greater than or equal to 50% by weight, in particular greater than or equal to 70% by weight, more particularly greater than or equal to 90% by weight, or even greater than or equal to 95% by weight, based on the total weight of the oleaginous substance.

Among the unsaturated fatty acids, the fatty acids selected in the group comprising:

undecen-10-oic acid (11/1),

hexadecen-9-oic acid (16/1, omega-7),

octadecen-9-oic acid (18/1, omega-9),

octadecen-11-oic acid (18/1, omega-7),

octadecadien-9,12-oic acid (18/2, omega-6),

octadecatrien-9,12,15-oic acid (18/3, omega-3),

gadoleic acid (20/1),

eicosatetraen-5,8,11,14-oic acid (20/4, omega-6),

eicosapentaen-5,8,11,14,17-oic acid (20/5, omega-3),

docosen-13-oic acid (22/1, omega-9),

docosahexaen-4,7,10,13,16,19-oic acid (22/6, omega-3),

tetracosen-15-oic acid (24/1, omega-9), and

a mixture thereof, may be cited. More particularly, the oleaginous substance may include a content in omega fatty acid or acids, more particularly omega-3, omega-6 and/or omega-9 fatty acids, greater than or equal to 50% by weight, more particularly greater than or equal to 75% by weight, more particularly greater than or equal to 90% by weight, or even greater than or equal to 99% by weight based on the total weight of the oleaginous substance.

Natural cyclodextrins (alpha, beta and gamma) are most of the time obtained from the bioconversion of maize starch by the bacterial enzyme cycloglycosyltransferase (CGTase).

These are cyclic oligosaccharides having respectively, for alpha, beta and gamma: 6, 7 or 8 alpha-D-glucopyranose units, the bonds connecting these units being of the alpha-(1,4)glucosidic type.

| | Alpha | beta | gamma |
|---|---|---|---|
| Number of glucopyranose units | 6 | 7 | 8 |
| Relative mass | 972 | 1,135 | 1,297 |
| Internal diameter (Å) | 4.7-5.2 | 6.0-6.4 | 7.5-8.3 |
| External diameter (Å) | 14.6 ± 0.4 | 15.4 ± 0.4 | 17.5 ± 0.4 |
| Depth (Å) | 7.9-8.0 | 7.9-8.0 | 7.9-8.0 |
| Solubility in water (g/100 mL, | 14.5 | 1.85 | 23.2 |
| 25° C.) | | | |

Cyclodextrin derivatives may be obtained by a substitution of primary or secondary hydroxyl groups, for example with alkyl radicals, particularly comprising 1 to 12 carbon atoms, for example methyl (—CH₃) or propyl (—C₃H₉) radicals. Such substitutions may make it possible to increase the lipophily of the cavity and increase the aqueous solubility of the cyclodextrin.

The structure of the cyclodextrins may be shown as a conical trunk with a hydrophobic cavity. The outside of the cyclodextrin molecule is generally hydrophilic, these are pseudo-amphiphilic molecules. Such pseudo-amphiphilic structure may allow the formation of inclusion complexes. The inclusion complex may have physico-chemical properties, which are independent from the guest molecule and thus improve the apparent water solubility of this molecule. This improved solubility may, for example, allow an improvement of the bioavalaibility of the molecule, particularly by improving dissolution rate of the molecule. The mixture of complexes according to the invention comprises at least two different cyclodextrins, which may each be present, in a content greater than or equal to 1% by weight, more particularly in a content greater than or equal to 10% by weight, or even in a content greater than or equal to 20% by weight, or even in a content greater than or equal to 30% by weight based on the total weight of the cyclodextrin.

In alternative, the mixture of complexes comprises two cyclodextrins, more particularly:

an alpha-cyclodextrin/beta-cyclodextrin mixture, more particularly in a ratio comprised between 10/1 and 1/10, or even between 4/1 and 1/4,

an alpha-cyclodextrin/gamma-cyclodextrin mixture, more particularly in a ratio comprised between 10/1 and 1/10, or even between 4/1 and 1/4, or

a beta-cyclodextrin/gamma-cyclodextrin mixture, more particularly in a ratio comprised between 10/1 and 1/10, or even between 4/1 and 1/4.

According to another alternative, the mixture of complexes comprises three cyclodextrins, more particularly an alpha-cyclodextrin/beta-cyclodextrin/gamma-cyclodextrin mixture, more particularly with an alpha-cyclodextrin/beta-cyclodextrin ratio comprised between 10/1 and 1/10, or even between 4/1 and 1/4, and/or with a beta-cyclodextrin/gamma-cyclodextrin ratio comprised between 10/1 and 1/10, or even between 4/1 and 1/4. According to another aspect, another object of the invention is a composition comprising or consisting in a mixture of at least two cyclodextrins selected among alpha-, beta- and gamma-cyclodextrin and/or derivatives thereof, and at least one oleaginous substance.

This composition may have an oleaginous substance/cyclodextrins weight ratio greater than or equal to 0.5, more particularly greater than or equal to 1, or even greater than or equal to 2. More particularly, the composition comprises a content in oleaginous substance greater than or equal to 10% by weight, in particular greater than or equal to 20% by weight, advantageously greater than or equal to 30% by weight, particularly greater than or equal to 40% by weight, more particularly greater than or equal to 50% by weight, most particularly greater than or equal to 60% by weight, or even greater than or equal to 70% by weight based on the total weight of the composition.

The composition may include at least two different cyclodextrins, each of these present in a content greater than or equal to 1% by weight, particularly in a content greater than or equal to 10% by weight, or even in a content greater than or equal to 20% by weight, or even in a content greater than or equal to 30% by weight based on the total weight of the cyclodextrin. According to a particular embodiment, the composition comprises inclusion complexes according to the invention, particularly with a content comprised between 1 and 99.9% by weight, more particularly comprised between 15 and 99% by weight, or even comprised between 25 and 95% by weight based on the total weight of the composition.

The composition according to the invention may be in the form of a liquid, particularly an aqueous liquid, a semisolid or a solid. It can more particularly be in the form of a powder, tablets, capsules, a cream, an emulsion, more particularly an aqueous or oily emulsion, or even a multiple emulsion, of liposomes, nanoparticles, microparticles or a suspension. The compositions according to the invention may be pharmaceutical, pharmafood, veterinary, nutraceutical, dietary or cosmetic compositions comprising a mixture of inclusion complexes according to the invention.

The inclusion of an oleaginous substance, and more particularly fatty acids, in particular polyunsaturated fatty acids, or triglycerides, salts and/or esters thereof in cyclodextrin mixtures, in accordance with the invention, may make it possible to obtain aqueous, solid or semisolid formulations, at 20° C. and at atmospheric pressure, containing this oleaginous substance, and particularly these polyunsaturated fatty acids and/or triglycerides, salts and esters thereof, while eliminating or strongly reducing the problems relating to the oxidizibility or instability thereof, as well as to reduce or eliminate their taste and/or their smell. Thus, another object of the invention is the use of a mixture of at least two cyclodextrins as stabilisation and/or taste and/or smell masking agents for an oleaginous substance, more particularly unsaturated fatty acids, in a composition, more particularly a dietary, nutraceutical, cosmetic, pharmaceutical, or veterinary composition, further comprising at least one oleaginous substance.

The invention also aims at using a mixture of at least two cyclodextrins and one oleaginous substance, particularly of at least one unsaturated fatty acid, for the preparation of a drug, especially one intended to treat or prevent cardiovascular diseases. The object of the invention is a method for preparing inclusion complexes comprising at least the steps consisting in:

solubilising at least two cyclodextrins selected from alpha-, beta- and gamma-cyclodextrin and/or derivatives thereof, more particularly in degassed water,

adding to this solution at least one oleaginous substance,

stirring the mixture, particularly in an inert atmosphere and/or in the absence of light, more particularly at a temperature comprised between 10 and 40° C.,

collecting the synthetized complexes.

The complexes may be directly collected in the form of an emulsion, on in the form of a powder, more particularly by lyophilisation of the emulsion or by spray drying.

### DETAILED DESCRIPTION

The following examples are given for illustration and are not limitative.

### Examples

### Example 1

### Inclusion Complexes of 60% Camelina Oil by a Binary Mixture of 40% Cyclodextrins Composed of 50% Alpha-Cyclodextrin and 50% β-Cyclodextrin

1 g of the mixture of cyclodextrins composed of 50% alpha-cyclodextrin and 50% beta-cyclodextrin is introduced into a vessel and 30 ml of degassed water are added. This is stirred on a plate rotating at 300 rpm to complete dissolution of the cyclodextrin mixture. 1.5 g of camelina oil is added, and constant stirring is maintained at 300 rpm away from direct light on the rotating plate for 24 hours, at room temperature. A stable white suspension composed of 40% of a binary mixture of cyclodextrin and 60% camelina oil forms. Upon completion of the lyophilisation, a rich powder containing 60% oil is collected with a yield of 76%. The solubility of the lyophilised complexes is then tested by putting them back into water, which results in an opalescent solution.

### Example 2

### Inclusion Complexes of 60% Camelina Oil by a Binary Mixture of 40% Cyclodextrins Composed of 30% α-Cyclodextrin and 10% β-Cyclodextrin

1 g of the mixture of cyclodextrins composed of 0.75 g of α-cyclodextrin and 0.25 g β-cyclodextrin plus 30 ml of degassed water is introduced into a vessel. This is stirred on a plate rotating at 300 rpm to complete dissolution of the cyclodextrin mixture. 1.5 g of camelina oil is then added, and the constant stirring at 300 rpm is maintained in the absence of light on a rotating plate for 24 hours, at room temperature. A stable white suspension composed of 40% of the binary mixture of cyclodextrin and 60% camelina oil forms. Upon completion of the lyophilisation, a rich powder containing 60% oil is collected with a yield of 75%. The solubility of the lyophilised complexes is tested by putting them back into water, which results in an opalescent solution.

### Example 3

### Inclusion Complexes of 70% Camelina Oil by a Binary Mixture of 30% Cyclodextrins Composed of 15% α-Cyclodextrin and 15% β-Cyclodextrin

0.75 g of the mixture of cyclodextrins composed of 0.375 g of α-cyclodextrin and 0.375 of β-cyclodextrin plus 30 ml of degassed water is introduced into a vessel. This is stirred on a plate rotating at 300 rpm to complete dissolution of the cyclodextrin mixture. 1.75 g of camelina oil is then added, and the constant stirring at 300 rpm is maintained away from direct light on the rotating plate for 24 hours, at room temperature. A stable white suspension, composed of 30% of a binary mixture of cyclodextrin and 70% camelina oil forms. Upon completion of the lyophilisation, a rich powder containing 70% oil is collected with a yield of 77%. The solubility of the lyophilised complexes is then tested by putting them back into water, which results in an opalescent solution.

### Example 4

### Inclusion Complexes of 70% Camelina Oil by a Binary Mixture of 30% Cyclodextrins Composed of 20% α-Cyclodextrin and 10% β-Cyclodextrin

0.75 g of the mixture of cyclodextrins composed of 0.5 g of α-cyclodextrin and 0.25 g of β-cyclodextrin, plus 30 ml of degassed water are added into a vessel. This is stirred on a plate rotating at 300 rpm to complete dissolution of the cyclodextrin mixture. 1.75 g of camelina oil is added, and the constant stirring is maintained at 300 rpm away from direct light on the rotating plate for 24 hours, at room temperature. A stable white suspension composed of 30% of binary mixture of cyclodextrin and 70% camelina oil forms. Upon completion of the lyophilisation, a rich powder containing 70% oil is collected with a yield of 77%. The solubility of the lyophilised complexes is then tested by putting them back into water, which results in an opalescent solution.

### Example 5

### Inclusion Complexes of 80% Camelina Oil by a Binary Mixture of 20% Cyclodextrins Composed of 10% α-Cyclodextrin and 10% β-Cyclodextrin

0.5 g of the mixture of cyclodextrins composed of 0.25 g of α-cyclodextrin and 0.25 g of β-cyclodextrin plus 30 ml of degassed water are introduced into a vessel. This is stirred on a plate rotating at 300 rpm to complete dissolution of the cyclodextrin mixture. 2 g of camelina oil are added, and the constant stirring is maintained at 300 rpm away from direct light on a rotating plate for 24 hours, at room temperature. A stable white suspension composed of 20% of the binary mixture of cyclodextrin and 80% camelina oil forms. Upon completion of the lyophilisation, a rich powder containing 80% oil is collected with a yield of 78%. The solubility of lyophilised complexes is then tested by putting them back into water, which results in an opalescent solution.

### Example 6

### Inclusion Complexes of 80% Camelina Oil by a Binary Mixture of 20% Cyclodextrins Composed of 15% α-Cyclodextrin and 5% β-Cyclodextrin

0.5 g of a mixture of cyclodextrins composed of 0.375 g of α-cyclodextrin and 0.125 g of β-cyclodextrin plus 30 ml of degassed water are introduced into a vessel. This is stirred on a plate rotating at 300 rpm to complete dissolution of the cyclodextrin mixture. 2 g of camelina oil are then added, and the stirring is maintained constant at 300 rpm away from direct light on a rotating plate for 24 hours, at room temperature. A stable white suspension composed of 20% of the binary mixture of cyclodextrin and 80% camelina oil forms. Upon completion of the lyophilisation, a rich powder containing 80% oil is collected with a yield of 76%. The solubility of the lyophilised complexes is then tested by putting them back into water, which results in an opalescent solution.

### Example 7

### Inclusion Complexes of 60% Camelina Oil by a Binary Mixture of 40% Cyclodextrins Composed of 20% α-Cyclodextrin and 20% γ-Cyclodextrin

1 g of the cyclodextrin mixture composed of 0.5 g of α-cyclodextrin and 0.5 g of γ-cyclodextrin plus 30 ml of degassed water are introduced into a vessel. This is then stirred on a plate rotating at 300 rpm to complete dissolution of the cyclodextrin mixture. 1.5 g of camelina oil is then added, and the stirring is maintained constant at 300 rpm away from direct light on a rotating plate for 24 hours, at room temperature. A stable white suspension composed of 40% of the binary mixture of cyclodextrin and 60% camelina oil forms. Upon completion of the lyophilisation, a rich powder containing 60% oil is collected with a yield of 74%. The solubility of the lyophilised complexes is then tested by putting them back into water, which results in an opalescent solution.

### Example 8

### Inclusion Complexes of 60% Camelina Oil by a Binary Mixture of 40% Cyclodextrins Composed of 30% α-Cyclodextrin and 10% γ-Cyclodextrin

1 g of a mixture of cyclodextrins composed of 0.75 g of α-cyclodextrin and 0.25 g of γ-cyclodextrin plus 30 ml of degassed water are introduced into a vessel. This is stirred on a plate rotating at 300 rpm to complete dissolution of the cyclodextrin mixture. 1.5 g of camelina oil is then added, and the stirring is maintained constant at 300 rpm away from direct light on a rotating plate for 24 hours, at room temperature. A stable white suspension composed of 40% of the binary mixture of cyclodextrin and 60% camelina oil forms. Upon completion of the lyophilisation, a rich powder containing 60% oil is collected with a yield of 75%. The solubility of the lyophilised complexes is then tested by putting them back into water, which results in an opalescent solution.

### Example 9

### Inclusion Complexes of 70% Camelina Oil by a Binary Mixture of 30% Cyclodextrins Composed of 15% α-Cyclodextrin and 15% γ-Cyclodextrin

0.75 g of the cyclodextrin mixture composed of 0.375 g of α-cyclodextrin and 0.375 g of γ-cyclodextrin plus 30 ml of degassed water are introduced into a vessel. Then, This is stirred on a plate rotating at 300 rpm to complete dissolution of the cyclodextrin mixture. 1.75 g of camelina oil is then added, and the stirring is maintained constant at 300 rpm away from direct light on a rotating plate for 24 hours, at room temperature. A stable white suspension composed of 30% the binary mixture of cyclodextrin and 70% camelina oil forms. Upon completion of the lyophilisation, a rich powder containing 70% oil is collected with a yield of 74%. The solubility of the lyophilised complexes is then tested by putting them back into water, which results in an opalescent solution.

### Example 10

### Inclusion Complexes of 70% Camelina Oil by a Binary Mixture of 30% Cyclodextrins Composed of 20% α-Cyclodextrin and 10% γ-Cyclodextrin

0.75 g of the cyclodextrin mixture composed of 0.5 g of α-cyclodextrin and 0.25 g of γ-cyclodextrin plus 30 ml of degassed water are introduced into a vessel. This is stirred on a plate rotating at 300 rpm to complete dissolution of the cyclodextrin mixture. 1.75 g of camelina oil is then added, and the stirring is maintained constant at 300 rpm away from direct light on a rotating plate for 24 hours, at room temperature. A stable white suspension composed of 30% of the binary mixture of cyclodextrin and 70% camelina oil forms. Upon completion of the lyophilisation, a rich powder containing 70% oil is collected with a yield of 75%. The solubility of the lyophilised complexes is then tested by putting them back into water, which results in an opalescent solution.

### Example 11

### Inclusion Complexes of 80% Camelina Oil by a Binary Mixture of 20% Cyclodextrins Composed of 10% α-Cyclodextrin and 10% γ-Cyclodextrin

0.5 g of a cyclodextrin mixture composed of 0.25 g of α-cyclodextrin and 0.25 g of γ-cyclodextrin plus 30 ml of degassed water are introduced into a vessel. This is stirred on a plate rotating at 300 rpm to complete dissolution of the cyclodextrin mixture. 2 g of camelina oil are then added, and the stirring is maintained constant at 300 rpm away from direct light on a rotating plate for 24 hours, at room temperature. A stable white suspension composed of 20% of the binary mixture of cyclodextrin and 80% camelina oil forms. Upon completion of the lyophilisation, a rich powder containing 80% oil is collected with a yield of 75%. The solubility of the lyophilised complexes is then tested by putting them back into water, which results in an opalescent solution.

### Example 12

### Inclusion Complexes of 80% Camelina Oil by a Binary Mixture of 20% Cyclodextrins Composed of 15% α-Cyclodextrin and 5% γ-Cyclodextrin

0.5 g of a mixture of cyclodextrin composed of 0.375 g of α-cyclodextrin and 0.125 g of γ-cyclodextrin plus 30 ml of degassed water are introduced into a vessel. This is stirred on a plate rotating at 300 rpm to complete dissolution of the cyclodextrin mixture. 2 g of camelina oil are then added, and the stirring is maintained constant at 300 rpm away from direct light on a rotating plate for 24 hours, at room temperature. A stable white suspension composed of 20% of the binary mixture of cyclodextrin and 80% camelina oil forms. Upon completion of the lyophilisation, a rich powder containing 80% oil is collected with a yield of 75%. The solubility of the lyophilised complexes is then tested by putting them back into water, which results in an opalescent solution.

### Example 13

### Inclusion Complexes of 60% Camelina Oil by a Ternary Mixture of 40% Cyclodextrins Composed of 15% α-Cyclodextrin, 10% β-Cyclodextrin and 15% γ-Cyclodextrin

1 g of the cyclodextrin mixture composed of 0.375 g of α-cyclodextrin, 0.25 g of β-cyclodextrin and 0.375 g of γ-cyclodextrin plus 30 ml of degassed water is introduced into a vessel. This is stirred on a plate rotating at 300 rpm to complete dissolution of the cyclodextrin mixture. 1.5 g of camelina oil is then added, and the stirring is maintained constant at 300 rpm away from direct light on a rotating plate for 24 hours, at room temperature. A stable white suspension composed of 40% of the binary mixture of cyclodextrin and 60% camelina oil forms. Upon completion of the lyophilisation, a rich powder containing 60% oil is collected with a yield of 75%. The solubility of the lyophilised complexes is then tested by putting them back into water, which results in an opalescent solution.

### Example 14

### Inclusion Complexes of 70% Camelina Oil by a Ternary Mixture of 30% Cyclodextrins Composed of 10% α-Cyclodextrin, 10% β-Cyclodextrin and 10% γ-Cyclodextrin

0.75 g of the mixture of cyclodextrins composed of 0.25 g of α-cyclodextrin, 0.25 g of β-cyclodextrin and 0.25 g of γ-cyclodextrin plus 30 ml of degassed water are introduced into a vessel. This is stirred on a plate rotating at 300 rpm to complete dissolution of the cyclodextrin mixture. 1.75 g of camelina oil is then added, and the stirring is maintained constant at 300 rpm away from direct light on a rotating plate for 24 hours, at room temperature. A stable white suspension composed of 30% of the binary mixture of cyclodextrin and 70% camelina oil forms. Upon completion of the lyophilisation, a rich powder containing 70% oil is collected with a yield of 75%. The solubility of the lyophilised complexes is then tested by putting them back into water, which results in an opalescent solution.

### Example 15

### Inclusion Complexes of 79% Camelina Oil by a Ternary Mixture of 21% Cyclodextrins Composed of 7% α-Cyclodextrin, 7% α-Cyclodextrin and 7% γ-Cyclodextrin

0.525 g of the mixture of cyclodextrins composed of 0.175 g of α-cyclodextrin, 0.175 g of β-cyclodextrin and 0.175 g of γ-cyclodextrin plus 30 ml of degassed water are introduced into a vessel. This is stirred on a plate rotating at 300 rpm to complete dissolution of the cyclodextrin mixture. 1.975 g of camelina oil is then added, and the stirring is maintained constant at 300 rpm away from direct light on a rotating plate for 24 hours, at room temperature. A stable white suspension composed of 21% of the binary mixture of cyclodextrin and 79% camelina oil forms. Upon completion of the lyophilisation, a rich powder containing 79% oil is collected with a yield of 75%. The solubility of the lyophilised complexes is then tested by putting them back into water, which results in an opalescent solution.

### Example 16

### Inclusion Complexes of 60% Argan Oil by a Binary Mixture of 40% Cyclodextrins Composed of 30% α-Cyclodextrin and 10% γ-Cyclodextrin

1 g of the cyclodextrin mixture composed of 0.75 g of α-cyclodextrin and 0.75 g of γ-cyclodextrin plus 30 ml of degassed water is introduced into a vessel. This is stirred on a plate rotating at 300 rpm to complete dissolution of the cyclodextrin mixture. Then, 1.5 g of argan oil is added, and the stirring is maintained constant at 300 rpm away from direct light on a rotating plate for 24 hours, at room temperature. A stable white suspension composed of 40% of the binary mixture of cyclodextrin and 60% argan oil forms. Upon completion of the lyophilisation, a rich powder containing 60% oil is collected with a yield of 77%. The solubility of the lyophilised complexes is then tested by putting them back into water, which results in an opalescent solution.

### Example 17

### Inclusion Complexes of 70% Argan Oil by a Binary Mixture of 30% Cyclodextrins Composed of 15% α-Cyclodextrin and 15% γ-Cyclodextrin

0.75 g of the cyclodextrin mixture composed of 0.375 g of α-cyclodextrin and 0.375 g of γ-cyclodextrin plus 30 ml of degassed water are introduced into a vessel. This is stirred on a plate rotating at 300 rpm to complete dissolution of the cyclodextrin mixture. Then, 1.75 g of argan oil is added, and the stirring is maintained constant at 300 rpm away from direct light on a rotating plate for 24 hours, at room temperature. A stable white suspension composed of 30% of the binary mixture of cyclodextrin and 70% argan oil forms. Upon completion of the lyophilisation, a rich powder containing 70% oil is collected with a yield of 73%. The solubility of the lyophilised complexes is then tested by putting them back into water, which results in an opalescent solution.

### Example 18

### Inclusion Complexes of 70% Argan Oil by a Binary Mixture of 30% Cyclodextrins composed of 20% α-Cyclodextrin and 10% γ-Cyclodextrin

0.75 g of the cyclodextrin mixture composed of 0.5 g of α-cyclodextrin and 0.25 g of γ-cyclodextrin plus 30 ml of degassed water are introduced in a vessel. This is stirred on a plate rotating at 300 rpm to complete dissolution of the cyclodextrin mixture. 1.75 g of argan oil is then added, and the stirring is maintained constant at 300 rpm away from direct light on a rotating plate for 24 hours, at room temperature. A stable white suspension composed of 30% of the binary mixture of cyclodextrin and 70% argan oil forms. Upon completion of the lyophilisation, a rich powder containing 70% oil is collected with a yield of 76%. The solubility of the lyophilised complexes is then examined by putting them back into water, which results in an opalescent solution.

### Example 19

### Inclusion Complexes of 60% Argan Oil by a Ternary Mixture of 40% Cyclodextrins Composed of 15% α-Cyclodextrin, 10% β-Cyclodextrin and 15% γ-Cyclodextrin

1 g of the cyclodextrin mixture composed of 0.375 g of α-cyclodextrin, 0.25 g of β-cyclodextrin and 0.375 g of γ-cyclodextrin plus 30 ml of degassed water are introduced into a vessel. This is stirred on a plate rotating at 300 rpm to complete dissolution of the cyclodextrin mixture. 1.5 g of argan oil is then added, and the stirring is maintained constant at 300 rpm away from direct light on a rotating plate for 24 hours, at room temperature. A stable white suspension composed of 40% of the binary mixture of cyclodextrin and 60% argan oil forms. Upon completion of the lyophilisation, a rich powder containing 60% oil is collected with a yield of 70%. The solubility of the lyophilised complexes is then tested by putting them back into water, which results in an opalescent solution.

### Example 20

### Inclusion Complexes of 70% Argan Oil by a Ternary Mixture of 30% Cyclodextrins Composed of 10% α-Cyclodextrin, 10% β-Cyclodextrin and 10% γ-Cyclodextrin

0.75 g of the cyclodextrin mixture composed of 0.25 g of α-cyclodextrin, 0.25 g of β-cyclodextrin and 0.25 g of γ-cyclodextrin plus 30 ml of degassed water are introduced in a vessel. This is stirred on a plate rotating at 300 rpm to complete dissolution of the cyclodextrin mixture. 1.75 g of argan oil is then added, and the stirring is maintained constant at 300 rpm away from direct light on a rotating plate for 24 hours, at room temperature. A stable white suspension composed of 30% of the binary mixture of cyclodextrin and 70% argan oil forms. Upon completion of the lyophilisation, a rich powder containing 70% oil is collected with a yield of 70%. The solubility of the lyophilised complexes is then tested by putting them back into water, which results in an opalescent solution.

### Example 21

### Inclusion Complexes of 79% Argan Oil by a Ternary Mixture of 21% Cyclodextrins Composed of 7% α-Cyclodextrin, 7% β-Cyclodextrin and 7% γ-Cyclodextrin

0.525 g of the cyclodextrin mixture composed of 0.175 g of α-cyclodextrin, 0.175 g of β-cyclodextrin and 0.175 g of γ-cyclodextrin plus 30 ml of degassed water are introduced into a vessel. This is stirred on a plate rotating at 300 rpm to complete dissolution of the cyclodextrin mixture. 1.975 g of argan oil is then added, and the stirring is maintained constant at 300 rpm away from direct light on a rotating plate for 24 hours, at room temperature. A stable white suspension composed of 21% of the binary mixture of cyclodextrin and 79% argan oil forms. Upon completion of the lyophilisation, a rich powder containing 79% oil is collected with a yield of 76%. The solubility of the lyophilised complexes is then tested by putting them back into water, which results in an opalescent solution.

The invention claimed is:

1-18. (canceled)

19. A mixture of inclusion complexes comprising:
at least two different cyclodextrins selected from alpha- beta- and gamma-cyclodextrin and/or derivatives thereof, in particular derivatives thereof modified by primary and/or secondary hydroxyl groups, and
at least one oleaginous substance, in particular selected from animal, vegetable and synthetic oils.

20. A mixture of complexes according to claim 19, further comprising a content of oleaginous substance greater than or equal to 10% by weight, particularly greater than or equal to 20% by weight, advantageously greater than or equal to 30% by weight, in particular greater than or equal to 40% by weight, more particularly greater than or equal to 50% by weight, particularly greater than or equal to 60% by weight, or even greater than or equal to 70% by weight based on the total weight of the complexes.

21. A mixture of complexes according to claim 19, wherein the oleaginous substance comprises at least one fatty acid, particularly a saturated and/or unsaturated fatty acid, a corresponding ester or triglyceride, in particular a monounsaturated or polyunsaturated fatty acid.

22. A mixture of complexes according claim 19, wherein the oleaginous substance comprises a content of unsaturated fatty acid greater than or equal to 30% by weight, particularly greater than or equal to 50% by weight, more particularly greater than or equal to 70% by weight, in particular greater than or equal to 90% by weight, or even greater than or equal to 95% by weight based on the total weight of the oleaginous substance.

23. A mixture of complexes according to claim 19, wherein the oleaginous substance comprises a content in omega fatty acid or acids, more particularly omega-3, omega-6 and/or omega-9 fatty acid or acids greater than or equal to 50% by weight, particularly greater than or equal to 75% by weight, more particularly greater than or equal to 90% by weight, or even greater than or equal to 99% by weight based on the total weight of the oleaginous substance.

24. A mixture of complexes according to claim 19, further comprising at least two cyclodextrins, each in a content greater than or equal to 1% by weight, more particularly in a content greater than or equal to 10% by weight, or even in a content greater than or equal to 20% by weight, or even in a content greater than or equal to 30% by weight based on the total weight of the cyclodextrin.

25. A mixture of complexes according to claim 19, further comprising two cyclodextrins, including:
an alpha-cyclodextrin/beta-cyclodextrin mixture, particularly in a ratio comprised between 10/1 and 1/10, or even between 4/1 and 1/4;
an alpha-cyclodextrin/gamma-cyclodextrin mixture, particularly in a ratio comprised between 10/1 and 1/10, or even between 4/1 and 1/4, or
a beta-cyclodextrin/gamma-cyclodextrin mixture, particularly in a ratio comprised between 10/1 and 1/10, or even between 4/1 and 1/4.

26. A mixture of complexes according to claim 19, further comprising three cyclodextrins, including an alpha-cyclodextrin/beta-cyclodextrin/gamma-cyclodextrin mixture, in particular with a ratio of alpha-cyclodextrin/beta-cyclodextrin comprised between 10/1 and 1/10, or even between 4/1 and 1/4, with an alpha-cyclodextrin/gamma-cyclodextrin ratio comprised between 10/1 and 1/10, or even between 4/1 and 1/4, and/or with a beta-cyclodextrin/gamma-cyclodextrin ratio comprised between 10/1 and 1/10, or even between 4/1 and 1/4.

27. A composition comprising a mixture of at least two cyclodextrins selected among alpha-, beta- and gamma-cyclodextrin and/or derivatives thereof, and at least one oleaginous substance.

28. A composition according to claim 27, wherein the oleaginous substance/cyclodextrins weight ratio is greater than or equal to 0.5, more particularly greater than or equal to 1, or even greater than or equal to 2.

29. A composition according to claim 27, wherein the oleaginous substance is greater than or equal to 40% by weight, more particularly greater than or equal to 50% by weight, in particular greater than or equal to 60% by weight, or even greater than or equal to 70% by weight based on the total weight of the composition.

30. A composition according to claim 27, further comprising at least two cyclodextrins, each in a content greater than or equal to 1% by weight, more particularly in a content greater than or equal to 10% by weight, or even in a content greater than or equal to 20% by weight, or even in a content greater than or equal to 30% by weight based on the total weight of the cyclodextrin.

31. A composition according to claim 27, further comprising inclusion complexes in a content comprised between 1 and 99.9% by weight, more particularly between 15 and 99% by weight, or even between 25 and 95% by weight based on the total weight of the composition.

32. A composition according to claim 27, wherein the composition is in the form of a powder, tablets, capsules, a cream, an emulsion, more particularly an aqueous or an oily emulsion, or even a multiple emulsion, of liposomes, nanoparticles, microparticles or a suspension.

33. A process for producing a composition, more particularly a dietary, nutraceutical, cosmetic, pharmaceutical, or veterinary composition, further comprising at least one oleaginous substance, comprising the step of mixing at least two cyclodextrins as taste/smell masking agent of oleaginous substances, including unsaturated fatty acids.

34. A process for preparing a drug, particularly intended for treating or preventing cardiovascular diseases, comprising the step of mixing at least two cyclodextrins and one oleaginous substance, including at least one unsaturated fatty acid.

35. A method for preparing inclusion complexes comprising at least the steps of:
solubilising at least two cyclodextrins selected from alpha-, beta- and gamma-cyclodextrin and/or derivatives thereof, more particularly in degassed water;
adding at least one oleaginous substance to this solution;
stirring the mixture, in an inert atmosphere and/or in the absence of light, in particular at a temperature comprised between 10 and 40° C.; and
collecting the complexes formed.

36. A method according to claim 35, further comprising directly collecting the complexes in the form of an emulsion, or in the form of a powder, more particularly by lyophilising the emulsion or by spray-drying.
